(19) United States
(12) Patent Application Publication
(10) Pub. No.: US 2012/0051502 A1
(43) Pub. Date: Mar. 1, 2012

(54) MAMMOGRAPHY SYSTEM

(75) Inventors: Yasunori OHTA, Kanagawa (JP); Wataru Ito, Kanagawa (JP); Masahiko Yamada, Kanagawa (JP); Takao Kuwabara, Kanagawa (JP); Takeshi Kamiya, Kanagawa (JP); Tetsuro Kusunoki, Kanagawa (JP)

(73) Assignee: FUJIFILM CORPORATION, Tokyo (JP)

(21) Appl. No.: 13/219,529

(22) Filed: Aug. 26, 2011

(30) Foreign Application Priority Data
Aug. 31, 2010 (JP) P2010-193882

(51) Int. Cl. A61B 6/04

(52) U.S. Cl. 378/37

(57) ABSTRACT

In a mammography system including a face guard, it is possible to prevent a human subject whose head is in close contact with the face guard from losing her posture and being in an unstable state. In a mammography system which includes a shield member for preventing radiation from being irradiated onto the face of the human subject, a radiation source which irradiates radiation is moved, such that, when radiation in two radiographing directions is irradiated from two focal positions distant from the chest wall of the human subject in a forward direction, the shield member is configured to be fixed at a predetermined position in the forward direction without being interlocking with the movement of the radiation source.

### BACKGROUND OF THE INVENTION

1. Field of the Invention

The present invention relates to a breast image radiographing device (hereinafter, referred to as a mammography system) which irradiates radiation beam onto the breast of a human subject from two different radiographing directions to detect radiological images in the radiographing directions, and in particular, to a mammography system which includes a face guard for preventing radiation from being irradiated onto the face of the human subject.

2. Description of the Related Art

In the related art, a system is known in which a plurality of images are displayed in combination, and stereoscopic view can be realized using parallax. An image (hereinafter, referred to as a stereoscopic image or a stereo image) which can be viewed stereoscopically is generated based on a plurality of images having parallax obtained by radiographing the same subject from different directions.

Moreover, such way of generating stereoscopic image is utilized not only in the field of digital cameras and televisions but also in the field of radiography. That is, a subject is irradiated with radiation beam from different directions, the radiation beam passing through the subject is detected by a radiological image detector to acquire plural radiological images having parallax, and a stereoscopic image is generated based on the radiological images. By generating a stereoscopic image in this way, a radiological image with a sense of depth can be observed and thereby more suitable radiological image for diagnosis can be observed.

As the above-described radiological image radiographing device which generates the stereoscopic image, a mammography radiographing device has been suggested. However, many existing mammography radiographing devices have a configuration in which radiation beam is irradiated in a direction along the chest wall of the human subject, that is, in a radiographing direction inclined left and right when viewed from the human subject. For this reason, a stereoscopic image to be displayed has a layout in which the chest wall is in the horizontal direction (JP2007-229201A).

In an interpretation protocol of a two-dimensional breast image in current medical practice, the breast image is displayed with a layout in which the chest wall is in the vertical direction. For this reason, there is a problem in that it is difficult to observe the breast image by comparison with the stereoscopic image having a layout in which the chest wall is in the horizontal direction.

Accordingly, for example, in a mammography radiographing device, it is considered that radiation beam is irradiated from a radiographing direction inclined back and forth when viewed from the human subject, thereby acquiring two radiological images.

In a mammography radiographing device of the related art which dadiographs a two-dimensional breast image, radiation beam is irradiated onto the breast from a radiation source unit provided above the human subject, and a face guard is configured with respect to the radiation source unit to prevent radiation irradiated from the radiation source unit from being irradiated onto the face of the human subject.

In many cases, a human subject who is forced to assume an unnatural posture because her breast is fixed brings her head into close contact with the face guard to maintain the unnatural posture.

In the above-described device which irradiates radiation beam from a radiographing direction inclined back and forth of the human subject to dadiograph a stereo image, it is considered that the face guard is configured with respect to the radiation source unit. In this case, it is necessary to move the radiation source unit back and forth when viewed from the human subject. For this reason, the face guard moves to a position distant forward from the human subject with the movement of the radiation source unit, such that the human subject cannot maintain her posture.

JP2004-188200A describes an X-ray tomosynthesis and mammography radiographing system in which radiation beam is irradiated from a radiographing direction inclined back and forth when viewed from the human subject, thereby acquiring a radiological image, but there is no suggestion on the above-described face guard.

### SUMMARY OF THE INVENTION

The present invention has been made in view of the above-mentioned problems and an object of the present invention is to provide a mammography system capable of ensuing that a human subject maintains her posture in a mammography system being provided with a face guard.

An aspect of the present invention provides a mammography system. The mammography system includes an irradiation unit which irradiates radiation beam from a plurality of different radiographing directions, a radiological image detector which detects radiological images in each radiographing directions through irradiation of the radiation beam from the irradiation unit, and a shield member which is configured to prevent the radiation from being irradiated onto the face of a human subject. The irradiation unit irradiates the radiation beam in the plurality of radiographing directions from a plurality of focal positions distant from the chest wall of the human subject in a forward direction by moving a radiation source which irradiates the radiation beam. The shield member is configured to be fixed at a predetermined position in the forward direction without interlocking with the movement of the radiation source when the radiation source moves shield member. The forward direction refers to the direction to which the chest wall of the human subject faces.

In the mammography system, the irradiation unit may include an arm portion which supports the radiation source movably in the forward direction, and the shield member may be coupled to the arm portion.

The mammography system may further include a compression plate which compress the breast. The shield member may be coupled to the compression plate.

The shield member may be expandable and contractible.

The shield member may be expandable and contractible, and may expand and contract while interlocking with the movement of the compression plate.

The mammography system may further includes a display unit which displays a stereoscopic image using two radiological images detected by the radiological image detector. The display unit may displays the stereoscopic image in which the extension direction of the chest wall of the human subject in the two radiological image is an up-down direction.

The extension of the chest wall means that the chest wall extends in the width direction.

According to the mammography system of the present invention, in a device which irradiates radiation beam in two radiographing directions from two focal positions distant from the chest wall of the human subject in the forward direction by moving the radiation source which irradiates the radiation beam, the shield member for preventing the radiation from being irradiated onto the face of the human subject is configured to be fixed at a predetermined position with respect to the forward direction without interlocking with the movement of the radiation source. Therefore, it is possible to prevent the human subject whose head is in close contact with the shield member from losing her posture and being in an unstable state.

### BRIEF DESCRIPTION OF THE DRAWINGS

FIG. 1 is a schematic configuration diagram of a breast image radiographing and display system using an embodiment of a mammography system of the present invention.

FIG. 2 is a diagram showing a state where a radiation source unit is moved in a breast image radiographing and display system shown in FIG. 1.

FIG. 3 is a block diagram showing the schematic internal configuration of a computer in a breast image radiographing and display system shown in FIG. 1.

FIG. 4 is a flowchart illustrating the action of a breast image radiographing and display system using an embodiment of a mammography system of the present invention.

FIGS. 5A and 5B are diagrams showing an example of a right-eye radiological image and a left-eye radiological image which are dadiographed such that a chest wall is in an up-down direction.

FIG. 6 is a schematic configuration diagram of a breast image radiographing and display system using another embodiment of a mammography system of the present invention.

FIG. 7 is a schematic configuration diagram of a breast image radiographing and display system using a further embodiment of a mammography system of the present invention.

FIG. 8 is a diagram showing an example of the configuration of a rod-type face guard.

### DESCRIPTION OF THE PREFERRED EMBODIMENTS

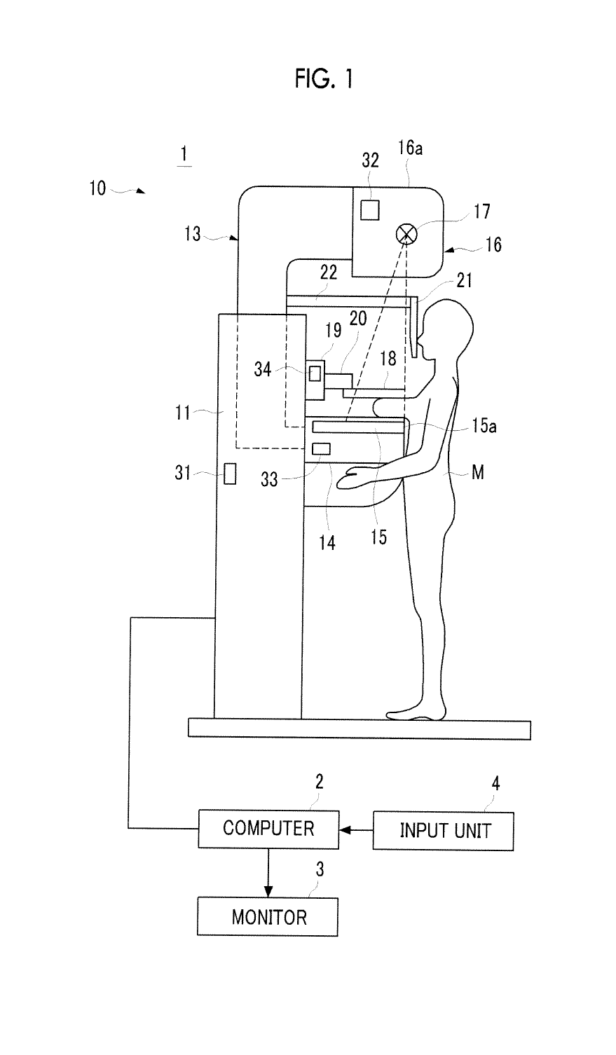

Hereinafter, a breast image radiographing and display system using an embodiment of a mammography system of the present invention will be described with reference to the drawings. FIG. 1 is a diagram showing the schematic configuration of the entire breast image radiographing and display system of this embodiment.

As shown in FIG. 1, a breast image radiographing and display system 1 of this embodiment includes a mammography system 10, a computer 2 which is connected to the mammography system 10, and a monitor 3 and an input unit 4 which are connected to the computer 2.

As shown in FIG. 1, the mammography system 10 includes a base 11, and an arm portion 13 which is supported movably in an up-down direction with respect to the base 11.

The arm portion 13 has a C shape. A radiographing stand 14 is attached to one end of the arm portion 13, and a radiation source unit 16 is attached to another end of the arm portion 13 to face the radiographing stand 14. The movement of the arm portion 13 in the up-down direction is controlled by an arm controller 31 which is incorporated in the base 11.

Inside the radiographing stand 14 are provided a radiological image detector 15, such as a flat panel detector, and a detector controller 33 which controls the reading of an electric charge signal from the radiological image detector 15, or the like.

Inside the radiographing stand 14 is also provided a circuit board on which a charge amplifier which converts an electric charge signal read from the radiological image detector 15 to a voltage signal, a correlated duplex sampling circuit which samples a voltage signal output from the charge amplifier, an AD detection unit which converts a voltage signal to a digital signal, and the like are provided.

The radiological image detector 15 can repeatedly record and read radiological images, and may be a so-called direct-type radiological image detector which directly receives radiation to generate electric charges or a so-called indirect-type radiological image detector which temporarily converts radiation to visible light and converts visible light to an electric charge signal. As a method of reading radiological image signals, it is preferable to use a so-called TFT reading method which turns on/off a TFT (thin film transistor) switch to read radiological image signals or a so-called light reading method which irradiates read light to read radiological image signals. The present invention is not limited thereto, and other methods may be used.

The radiation source unit 16 includes a radiation source 17, a radiation source controller 32, and a unit casing 16a in which the radiation source 17 and the radiation source controller 32 are accommodated. The radiation source controller 32 controls the timing at which radiation beam is irradiated from the radiation source 17 and the radiation beam generation conditions (tube current, time, tube current-time product, and the like) in the radiation source 17.

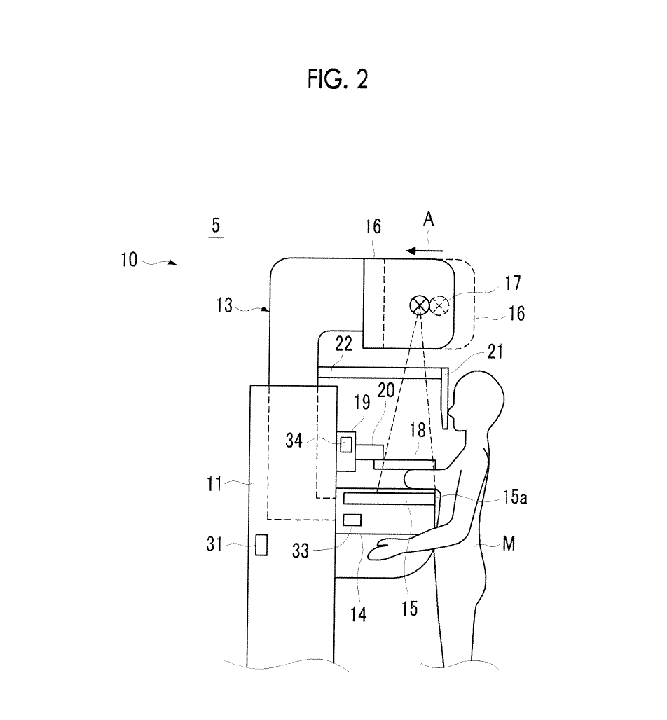

As shown in FIG. 2, the radiation source unit 16 is attached to the arm portion 13 movably in the forward direction (arrow A direction) from the chest wall of a human subject M with respect to the horizontal direction. The radiation source unit 16 moves in the forward direction from the chest wall of the human subject M, such that the radiation source 17 accommodated in the radiation source unit 16 moves together. The movement of the radiation source unit 16 is also controlled by the arm controller 31.

In this embodiment, at the time of radiographing two radiological images, the arm controller 31 moves the radiation source unit 16 such that, after a first radiological image is dadiographed in a state where the radiation source unit 16 is arranged near the chest wall of the human subject, the radiation source unit 16 is moved distant from the chest wall of the human subject, and a second radiological image is dadiographed.

In the central portion of the arm portion 13 are provided a compression plate 18 which is arranged above the radiographing stand 14 and compresses a breast, a support portion 20 which supports the compression plate 18, and a moving mechanism 19 which moves the support portion 20 in the up-down direction. The position of the compression plate 18 and the compressing pressure are controlled by a compression plate controller 34.

In the central portion of the arm portion 13, a face guard 21 is configured between the radiation source unit 16 and the compression plate 18 to prevent radiation irradiateted from the radiation source 17 in the radiation source unit 16 from being irradiated onto the face of the human subject M. The face guard 21 is attached to the arm portion 13 through a support member 22.

The face guard 21 is formed of a radiation-absorbent member, such as lead or copper, and is configured to be fixed at a predetermined position by the support member 22 without interlocking with the movement of the radiation source unit 16.

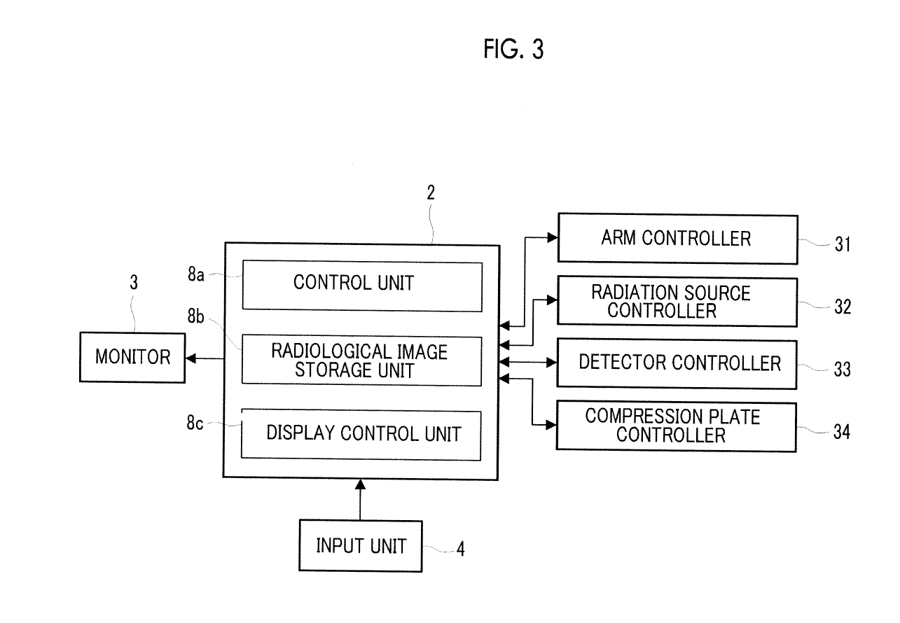

The computer 2 includes a central processing unit (CPU), a storage device, such as a semiconductor memory, a hard disk, or an SSD, and the like, which constitute a control unit 8a, a radiological image storage unit 8b, and a display control unit 8c shown in FIG. 3.

The control unit 8a outputs a predetermined control signal to various controllers 31 to 35, and performs overall control of the system. A specific control method will he described below.

The radiological image storage unit 8b stores two radiological image signals detected by the radiological image detector 15 through radiographing from two different radiographing directions in advance.

The display control unit 8c performs predetermined signal processing on radiological image signal read from the radiological image storage unit 8b and controls the monitor 3 to display the stereo image of the breast.

The input unit 4 is, for example, a keyboard or a pointing device, such as a mouse, and receives an input of radiographing conditions, a radiographing start instruction, or the like by the dadiographer.

The monitor 3 is configured to display a stereo image using two radiological image signals output from the computer 2. As a configuration in which a stereo image is displayed, for example, a configuration may be used in which radiological images based on two radiological image signals are respectively displayed on two screen, and a half minor, a polarizing glass, or the like is used such that one radiological image is incident on the right eye of the observer and another radiological image is incident on the left eye of the observer, thereby displaying a stereo image. A configuration may be made in which two radiological images are superimposingly displayed while shifting by a predetermined parallax amount and observed by a polarizing glass thereby generating a stereo image. A configuration may be made in which, like a parallax barrier method and a lenticular method, two radiological images are displayed on a 3D liquid crystal device which can be viewed stereoscopically, thereby generating a stereo image.

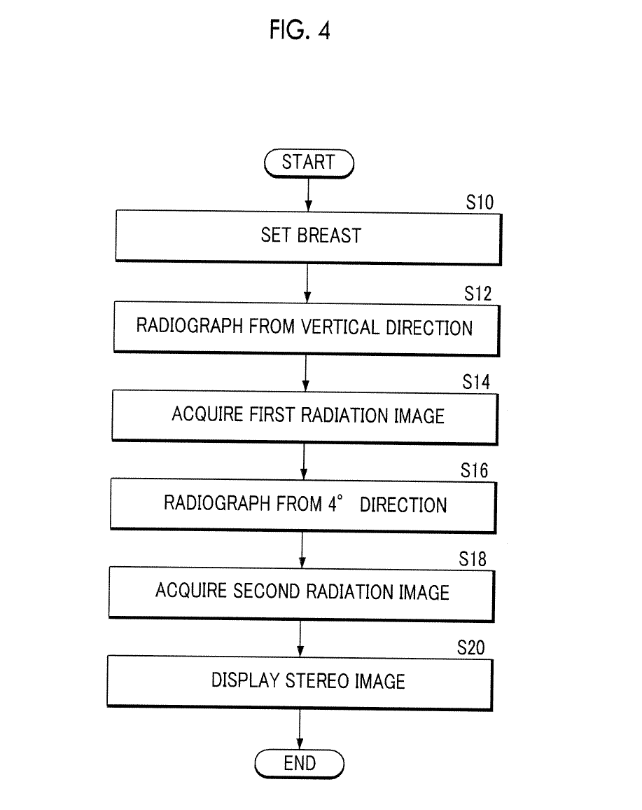

Next, the action of the breast image radiographing and display system of this embodiment will be described with reference to a flowchart shown in FIG. 4.

First, the breast of the human subject M is set on the radiographing stand 14, and the breast is compressed at a predetermined pressured by the compression plate 18 (S10).

If the dadiographer inputs a radiographing start instruction using the input unit 4, a first radiological image from two radiological images constituting a stereo image is dadiographed (S12).

Specifically, as shown in FIG. 1, the radiation source unit 16 is provided in front of the breast. In this state, first, the first radiological image is dadiographed. In this embodiment, at this time, the radiation source unit 16 is provided such that the line which connects the focal position of the radiation source 17 and the end surface 15a of the radiological image detector 15 is substantially perpendicular to the detection surface of the radiological image detector 15.

In a state where the radiation source unit 16 is arranged in the above-described manner. the control unit 8a outputs a control signal to the radiation source controller 32 and the detector controller 33 to irradiate radiation beam and to read radiological image signals. With the control signal, radiation beam is irradiateted from the radiation source 17, and a radiological image obtained by radiographing the breast from the 0° direction is detected by the radiological image detector 15. The radiological image signal is read by the detector controller 33 and stored in the radiological image storage unit 8b of the computer 2 after having been subjected to predetermined signal processing (S14).

Next, the control unit 8a reads a convergence angle θ for radiographing a stereo image set in advance, and outputs information regarding the read convergence angle θ to the arm controller 31. Although in this embodiment, θ0=4° is stored in advance as information regarding the convergence angle θ, the present invention is not limited thereto, and the dadiographer may set an arbitrary convergence angle using the input unit 4.

As shown in FIG. 2, the arm controller 31 outputs a control signal such that the radiation source unit 16 moves in a direction distant from the chest wall of the human subject. The radiation source unit 16 moves to a position where the line which connects the focal position of the radiation source 17 and the end surface 15 of the radiological image detector 15 is inclined at 4° with respect to the detection surface of the radiological image detector 15.

At this time, the face guard 21 is fixed at a predetermined position without interlocking with the movement of the radiation source unit 16.

In a state where the radiation source unit 16 is arranged at the above-described position, the second radiological image is dadiographed (S16).

Specifically, the control unit 8a outputs a control signal to the radiation source controller 32 and the detector controller 33 to irradiate radiation beam and to read radiological images. With the control signal, radiation beam is irradiateted from the radiation source 17, a radiological image obtained by radiographing the breast from the 4° direction is detected by the radiological image detector 15, and a radiological image signal is read by the detector controller 33 and stored in the radiological image storage unit 8b of the computer 2 after having been subjected to predetermined signal processing (S18).

The two radiological image signals stored in the radiological image storage unit 8b are output to the display control unit 8c. In the display control unit 8c, the radiological image signals are output to the monitor 3 after having been subjected to predetermined processing. In the monitor 3, the stereo image of the breast is displayed (S20).

According to the breast image radiographing and display system of this embodiment, the face guard 21 is configured to be fixed at a predetermined position with respect to the forward direction without interlocking with the movement of the radiation source 17. Therefore, it is possible to prevent the human subject whose head is in close contact with the face guard 21 from losing her posture and being in an unstable state.

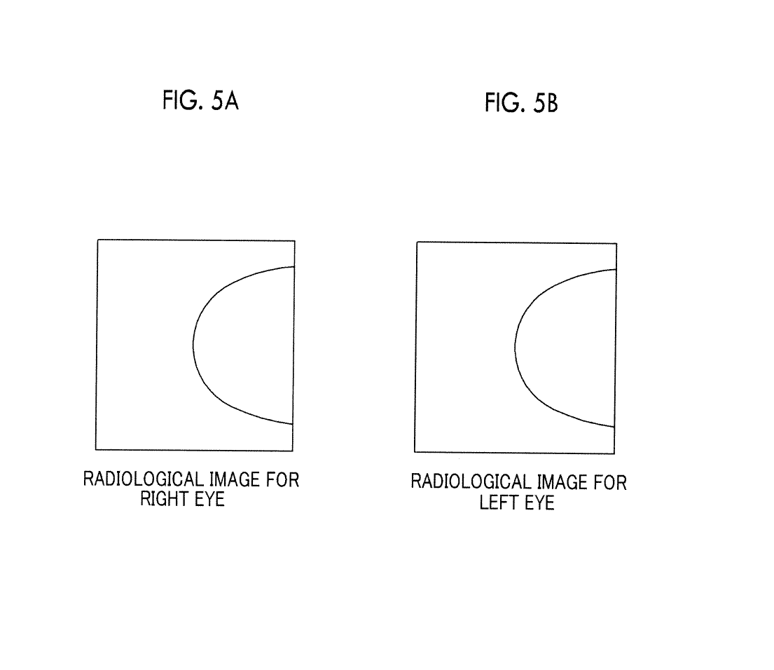

In the breast image radiographing and display system of this embodiment, radiation beam in two radiographing directions is irradiated from two focal positions distant from the chest wall of the human subject in the forward direction to dadiograph two radiological images. Thus, as shown in FIGS. 5A and 5B, it is possible to display a stereo image in which the extension direction of the chest wall is the up-down direction at the time of display. If a stereo image is displayed in the above-described manner, the stereo image can have the same direction as the display at the time of interpretation of a two-dimensional breast image in the related art, making it possible to easily compare the stereo image and the two-dimensional breast image.

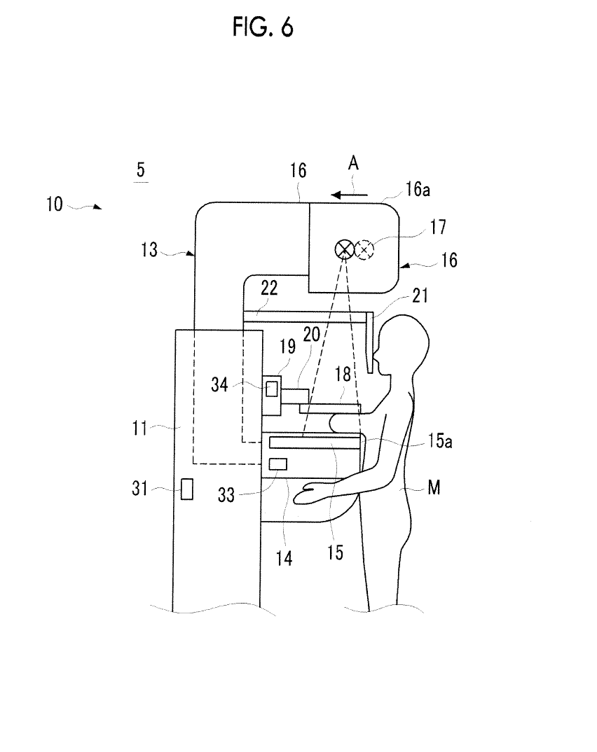

Although in the above-described embodiment, the entire radiation source unit 16 is moved to dadiograph two radiological images, the present invention is not limited thereto. For example, as shown in FIG. 6, while the unit casing 16a of the radiation source unit 16 is fixed, the radiation source 17 in the unit casing 16a may be moved in the direction distant from the chest wall of the human subject M.

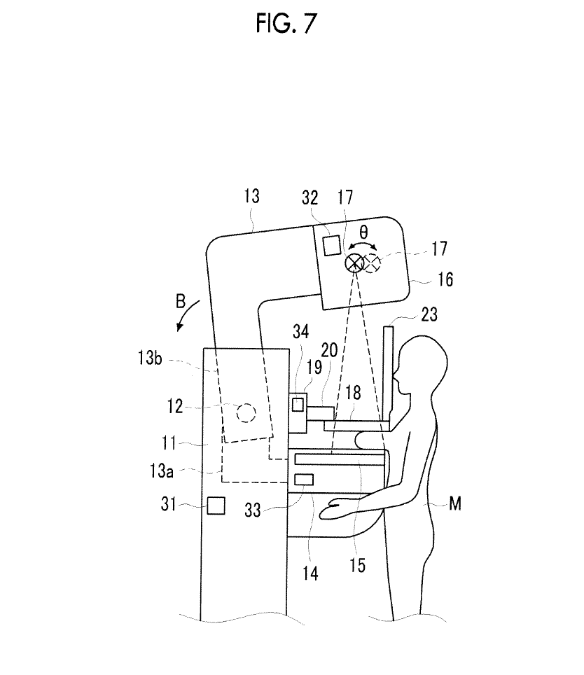

Although in the above-described embodiment, the face guard 21 is configured in the arm portion 13 through the support member 22, the present invention is not limited thereto. As shown in FIG. 7, a face guard 23 may be provided in the compression plate 18. Specifically, the face guard 23 may be extended in the vertical direction on the upper surface of the end portion of the compression plate 18 on the chest wall side.

As shown in FIG. 7, when the face guard 23 is configured in the compression plate 18, for example, as shown in FIG. 7, the arm portion 13 may have a support arm portion 13a which is movable only in the up-down direction, and a rotation arm portion 13b which is connected to the support arm portion 13a rotatably in an arrow B direction around a rotation shaft 12.

The rotation arm portion 13b may rotate around the rotation shaft 12 by the convergence angle θ, such that the radiation source 17 of the radiation source unit 16 may be moved in the forward direction (the direction distant) from the chest wall of the human subject NI to dadiograph two radiological images.

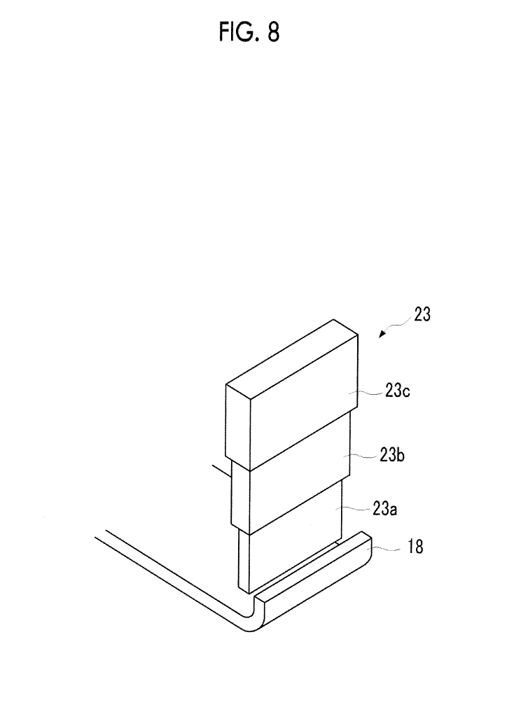

As described above, as shown in FIG. 8, the face guard 23 which is configured in the compression plate 18 may have a first member 23a. a second member 23b, and a third member 23c, and may be of an expandable and contractible rod type with the members nested. The expansion and contraction of the rod-type face guard 23 may be carried out manually or electrically. When the expansion and contraction may be carried out electrically, for example, the expansion and contraction may be carried out in accordance with the movement of the radiation source unit 16 or the radiation source 17.

Specifically, when the radiation source unit 16 or the radiation source 17 is distant from the chest wall of the human subject, a larger amount of radiation is irradiated onto the face of the human subject. At this time, a configuration may be made in which, in a state where the face guard 23 expands, as the radiation source unit 16 or the radiation source 17 is moved in the direction distant from the chest wall of the human subject, the face guard 23 gradually contracts electrically.

The face guard 23 may expand and contract in accordance with the movement of the compression plate 18. Specifically, when the compression plate 18 moves in the down direction, the face guard 23 expands, and when the compression plate 18 moves in the up direction, the face guard 23 contracts. Thus, the face guard 23 can be constantly in front of the face of the human subject, regardless of the up-down movement of the compression plate, thereby reducing the exposure to the human subject.

The above-described configuration can be realized, for example, using a rack-and-pinion mechanism or a cylinder mechanism using a hydraulic compress ure or the like.

The configuration in which the face guard is expandable and contractible is not limited to the above-described rod type. For example, an accordion type or a folding type may be used.

The configuration in which the face guard is expandable and contractible is not limited to a case where the face guard is configured in the compression plate 18, and as shown in FIG. 1, may be used when the face guard 21 is configured in the arm portion 13 through the support member 22.

In the breast image radiographing and display system of the above-described embodiment, a radiological image is dadiographed by irradiating radiation from the front direction of the breast as one radiological image from two radiological images constituting a stereo image. Therefore, the radiological image in the front direction may be used when a normal two-dimensional breast image is observed.

Although in the above-described embodiment, the mammography system of the present invention is applied to the breast stereo image radiographing device, the present invention is not limited thereto. The present invention may be applied to other devices insofar as the devices may dadiograph radiological images in a plurality of radiographing directions by moving the radiation source with respect to the forward direction from the chest wall of the human subject. For example, the present invention may be applied to a tomosynthesis radiographing device.

What is claimed is:

1. A mammography system comprising:
an irradiation unit which irradiates radiation beam from a plurality of different directions;
a radiological image detector which receives the radiation beam from the irradiation unit and captures images in each radiographing directions;
a shield member which is configured to prevent the radiation from being irradiated onto the face of a human subject,
wherein the irradiation unit irradiates the radiation beam from a plurality of focal positions distant from the chest wall of the human subject in a forward direction by moving a radiation source which irradiates the radiation, and
the shield member is configured to be fixed at a predetermined position in the forward direction when the radiation source moves.

2. The mammography system according to claim 1,
wherein the irradiation unit includes an arm portion which supports the radiation source movably in the forward direction, and
the shield member is configured in the arm portion.

3. The mammography system according to claim 1, further comprising:
a compression plate which compresses the breast of the human subject,
wherein the shield member is coupled to the compression plate.

4. The mammography system according to claim 1,
wherein the shield member is extendable and contractible.

5. The mammography system according to claim 2,
wherein the shield member is extendable and contractible.

6. The mammography system according to claim 3,
wherein the shield member is extendable and contractible.

7. The mammography system according to claim 3,
wherein the shield member is extendable and contractible, and configured to be extended and contracted interlocking to the movement of the compression plate.

8. The mammography system according to claim 1, further comprising:
a display unit which displays a stereoscopic image using a plurality of radiological images captured by the detector,
wherein the display unit displays the stereoscopic image in which the extension direction of the chest wall of the human subject in the plurality of radiological images is an up-down direction.

9. The mammography system according to claim 2, further comprising:
a display unit which displays a stereoscopic image using a plurality of radiological images captured by the detector,
wherein the display unit displays the stereoscopic image in which the extension direction of the chest wall of the human subject in the plurality of radiological images is an up-down direction.

10. The mammography system according to claim 3, further comprising:
a display unit which displays a stereoscopic image using a plurality of radiological images captured by the detector,
wherein the display unit displays the stereoscopic image in which the extension direction of the chest wall of the human subject in the plurality of radiological images is an up-down direction.

11. The mammography system according to claim 4, further comprising:
a display unit which displays a stereoscopic image using a plurality of radiological images detected by the detector,
wherein the display unit displays the stereoscopic image in which the extension direction of the chest wall of the human subject in the plurality of radiological images is an up-down direction.

12. The mammography system according to claim 5, further comprising:
a display unit which displays a stereoscopic image using a plurality of radiological images detected by the radiological image detector,
wherein the display unit displays the stereoscopic image in which the extension direction of the chest wall of the human subject in the plurality of radiological images is an up-down direction.

13. The mammography system according to claim 6, further comprising:
a display unit which displays a stereoscopic image using a plurality of radiological images detected by the radiological image detector,
wherein the display unit displays the stereoscopic image in which the extension direction of the chest wall of the human subject in the plurality of radiological images is an up-down direction.

14. The mammography system according to claim 7, further comprising:
a display unit which displays a stereoscopic image using a plurality of radiological images detected by the radiological image detector,
wherein the display unit displays the stereoscopic image in which the extension direction of the chest wall of the human subject in the plurality of radiological images is an up-down direction.
